(19) United States
(12) Patent Application Publication
(10) Pub. No.: US 2006/0276705 A1
(43) Pub. Date: Dec. 7, 2006

(54) Disposable tip cover for a tonometry apparatus

(76) Inventors: John R. Prisco, Jacksonville, FL (US); Joel A. Hembrock, Jacksonville, FL (US); John C. Bruce, Jacksonville, FL (US)

Correspondence Address: HODGSON RUSS LLP, ONE M & T PLAZA, SUITE 2000, BUFFALO, NY, 14203-2391, US

(21) Appl. No.: 11/144,159

(22) Filed: Jun. 3, 2005

(51) Int. Cl. A61B 3/16

(52) U.S. Cl. 600/406

(57) ABSTRACT

A disposable cover for a contact tonometer probe tip. The cover includes a tubular body and a cover tip. The tubular body is defined by a wall having an outer surface and an inner surface, and defines distal, proximal, and intermediate sections. The distal section terminates at a distal end, and the proximal section terminates at an open, proximal end. The proximal section forms a radial channel configured to mate with a corresponding feature of a probe tip and is characterized by an increased inner diameter. The outer surface along the intermediate section defines a conical shape that decreases in diameter from the proximal section to the distal section. Finally, the cover tip is formed at the distal end of the tubular body such that the cover is closed at the distal end.

### BACKGROUND

The present invention relates generally to the field of ophthalmological tonometers. More particularly, embodiments relate to a disposable cover for a contact tonometer probe tip.

A tonometer is an ophthalmological instrument for testing intraocular pressure. Such a test is commonly performed as part of a standard eye examination to detect the early stages of glaucoma. Glaucoma is characterized by an increased pressure within the eye that causes visual defects and ultimately may cause blindness. Because a patient suffering from glaucoma seldom experiences any symptoms until major damage occurs, regular testing is essential to detect glaucoma in the early stages before the retinal field is seriously diminished or ocular nerve damage has occurred.

One type of tonometer is known as a “contact” tonometer; one common form of this type of tonometer is an applanation tonometer. The contact tonometer includes a probe tip that is directly touched onto the surface of the patient's eye to form an indentation. The amount of force required to produce a given indentation is measured, and is used to determine intraocular pressure. More particularly, the resiliency of the eye's surface and the internal pressure of the eyeball resist the flattening or indentation imparted by the probe tip, and such force may then be converted to a measurement of intraocular pressure.

A variety of disease pathogens can be found on the eye surface, and especially in the fluid film that covers the eye. These pathogens include those that relate directly to the eye, such as conjunctivitis, and those that are systemic, such as immune system disorders. A contact tonometer must touch the eye, and therefore, may pose a risk of transferring such pathogens from one patient to another, or from patient to healthcare provider. A more preferred approach to address this concern is to place a disposable cover over the probe tip prior to each application. The cover is disposed of after each measurement, and helps to protect the eye from injury or irritation.

Contact tonometers continue to be highly valuable tools for detecting the early stages of glaucoma, and for performing intraocular eye pressure measurements for other reasons. To this end, disposable covers are essentially required when using a contact tonometer on multiple patients. However, known disposable covers are subject to possible deficiencies. Therefore, a need exists for an improved disposable cover for a probe tip of a contact tonometer providing improved mounting and minimal tonometer operational impediments.

### SUMMARY

Embodiments of the present invention relate to a disposable cover for a probe tip of a contact tonometer. The cover includes a tubular body and a cover tip. The tubular body is defined by a wall having an outer surface and an inner surface, with the tubular body defining a distal section, a proximal section, and an intermediate section. The distal section terminates at a distal end. The proximal section is positioned opposite the distal end and terminates in an open, proximal end. Further, the inner wall along the proximal section forms a radial channel longitudinally spaced from the proximal end. The radial channel is configured to mate with a corresponding feature of a probe tip and is characterized by an increased diameter as compared to an inner diameter of the tubular body immediately adjacent to the radial channel. The intermediate section extends between the distal and proximal sections. The outer surface along the intermediate section defines a conical shape that decreases in diameter from the proximal section to the distal section. Finally, the cover tip is formed at the distal end of the tubular body such that the cover is closed at the distal end. With this configuration, the disposable cover can be firmly secured to a probe tip in a proper location. In one embodiment, the disposable cover further includes a uniform thickness flange radially extending from the proximal end of the tubular body.

Other embodiments of the present invention relate to a combination contact tonometer and disposable cover. The contact tonometer includes a probe tip. The disposable cover includes a tubular body and a cover tip. The tubular body is defined by a wall having an outer surface and an inner surface. The tubular body further defines a distal section, a proximal section, and an intermediate section. The distal section terminates at a distal end. The proximal section is provided opposite the distal end and terminates in an-open, proximal end. The proximal section further forms a radial channel longitudinally spaced from the proximal end. The intermediate section extends between the distal and proximal sections, defining a conical shape along the outer surface thereof. More particularly, the outer surface at the intermediate section tapers in diameter from the proximal section to the distal section. Finally, the cover tip is formed at the distal end, such that the cover is closed at the distal end. With this in mind, the combination tonometer and cover are configured such that the cover is secured over the probe tip by capturing a feature of the probe tip within the radial channel in a manner allowing subsequent removal of the cover from the probe tip. In one embodiment, the probe tip includes a frame maintaining a sensor, with the frame including a radially extending engagement feature. With this configuration, upon assembly of the cover to the probe tip, the engagement feature nests within the radial channel. In another embodiment, the probe tip includes a frame maintaining a sensor such that the sensor can move relative to the frame between an activated position in which a portion of the sensor projects distally from the frame, and a deactivated position in which the sensor is recessed within the frame. With this in mind, the cover is configured to maintain a spacing in between the cover tip and the distal end of the frame when the sensor is in the deactivated position. In yet another embodiment, the tonometer further includes a detection device adapted to detect insertion of the cover over the probe tip.

### BRIEF DESCRIPTION OF THE DRAWINGS

FIG. 1 is a side view of one embodiment of a disposable cover for a contact tonometer in accordance with the present invention;

FIG. 2A is a cross-sectional view of the disposable cover of FIG. 1;

FIG. 2B is an enlarged view of a portion of the cross-sectional view of FIG. 2A;

FIG. 2C is an enlarged view of another portion of the cross-sectional view of FIG. 2A;

FIG. 3 is an exploded view of the cover of FIG. 1 in combination with an insertion tool and a contact tonometer;

FIG. 4 is a perspective view of the cover/tonometer of FIG. 3 upon assembly;

FIG. 5A is an enlarged, cross-sectional view of the disposable cover of FIG. 1 applied to a probe tip portion of the tonometer of FIG. 3, with the probe tip in a deactivated position;

FIG. 5B is a cross-sectional view of the cover and probe tip of FIG. 5B, with the probe tip in an activated position;

FIG. 6 is an exploded, perspective view of another embodiment disposable cover in conjunction with a contact tonometer probe tip;

FIG. 7A is a side view of the cover of FIG. 1 partially assembled to an another embodiment tonometer in accordance with the present invention; and

FIG. 7B is a side view of the cover/tonometer of FIG. 7A upon final assembly.

### DETAILED DESCRIPTION

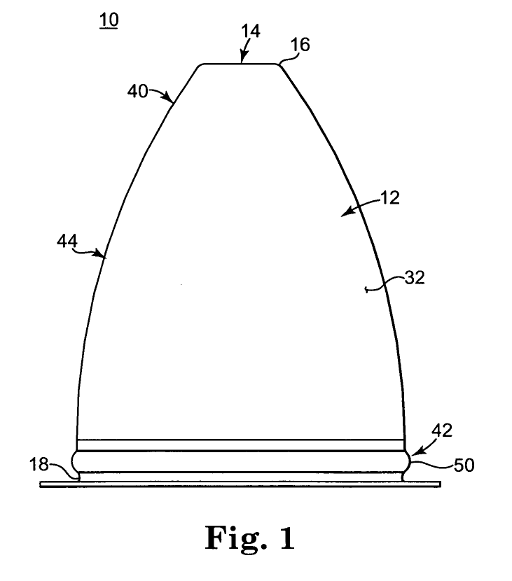

One embodiment of a disposable cover 10 for a contact tonometer probe tip is shown in FIG. 1. The cover 10 includes a tubular body 12 and a cover tip 14. In general terms, the cover tip 14 closes a distal end 16 of the tubular body 12, with a proximal end 18 of the tubular body 12 being open (it being understood that the terms “distal” and “proximal” are with respect to an orientation of the cover 10 when applied to a tonometer and thus relative to an operator handling the tonometer). With this configuration, the cover 10 can be inserted over a probe tip (not shown) via the open proximal end 18, with the cover tip 14 providing a protective surface for sensor components of the probe tip. As described in greater detail below, the cover 10 is adapted to consistently establish a secured fit over the probe tip with minimal interference to probe operations.

In one embodiment, the cover 10 is a unitary, homogenous structure, formed from a material approved for contact with the human eye. In one embodiment, the cover 10 is formed from a hypoallergenic material such as a synthetic elastomer as described in U.S. patent application Ser. No. 10/923,312 (filed Aug. 20, 2004), the teachings of which are incorporated herein by reference. Alternatively, a wide variety of other materials can be employed, such as medical grade polyurethane, low density polyethylene, polypropylene, etc. The cover 10 can be unitarily formed using a variety of techniques, such as thermoforming (extruded film process), injection molding, etc. Alternatively or in addition, portions of the cover 10 can be separately formed and subsequently assembled.

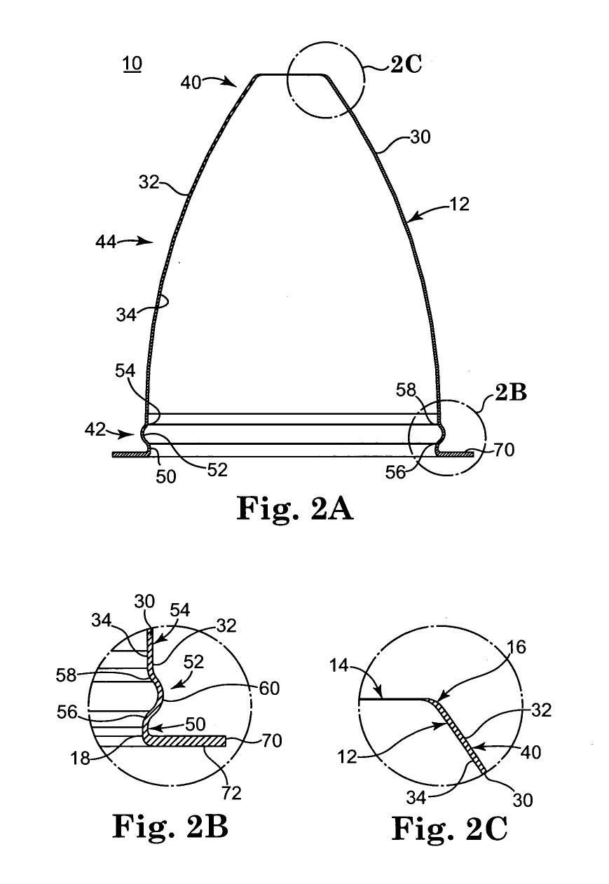

With additional reference to FIG. 2A, the tubular body 12 includes a wall 30 having an outer surface 32 and an inner surface 34. Further, the tubular body 12 can be characterized as generally defining a distal section 40, a proximal section 42, and an intermediate section 44. The distal section 40 terminates at the distal end 16, whereas the proximal section 42 terminates at the open, proximal end 18. Finally, the intermediate section 44 extends between the distal and proximal sections 40, 42. In one embodiment, the wall 30 has a thickness on the order of about 0.002-0.010 inch; more preferably about 0.005 inch. Alternatively, other dimensions can be employed. Regardless, the wall 30 is preferably characterized by a substantially uniform thickness (i.e., thickness varies by no more than 0.01 inch along a longitudinal length of the wall 30), it being understood that the selected manufacturing technique will affect the achievable uniformity.

The proximal section 42 forms or defines a base region 50, a radial channel 52, and a transition region 54. The radial channel 52 extends between the base and transition regions 50, 54, and is adapted to facilitate mating with a corresponding feature of a contact tonometer probe tip (not shown) as described below. In particular, and with additional reference to FIG. 2B, the radial channel 52 is formed by a longitudinal curve defined by the inner surface 34 of the wall 30. The base region 50 longitudinally spaces the radial channel 52 from the proximal end 18, whereas the transition region 54 extends longitudinally from the radial channel 52 to the intermediate section 44. To this end, an intersection of the radial channel 52/base region 50 can be characterized as defining a first point 56, and an intersection of the radial channel 52/transition region 54 can be characterized as defining a second point 58. With these conventions in mind, the curvature of the inner surface 34 along the radial channel 52 extends from the first point 56 to the second point 58.

In forming the radial channel 52, the inner surface 34 longitudinally increases in diameter from the first point 56 to a middle region 60 of the radial channel 52, and longitudinally decreases in diameter from the middle region 60 to the second point 58. With this configuration, then, a maximum inner diameter of the proximal section 42 is defined at the middle region 60 of the radial channel 52. In one embodiment, the wall 30 has a substantially uniform thickness along at least the proximal section 42, such that the outer surface 32 defines the same curvature as the inner surface 34 at the radial channel 52. In addition, and in one embodiment, an inner diameter of the tubular body 12 at the first point 56 is less than the inner diameter of the tubular body 12 at the second point 58; further, an inner diameter of the base region 50 is less than an inner diameter of the transition region 54, with the transition region 54 having a substantially uniform inner diameter. With this configuration, and as described in greater detail below, the reduced-diameter base region 50 facilitates a secure fit against the contact tonometer probe tip (not shown) upon final assembly.

In one embodiment, the cover 10 further includes a flange 70 as best shown in FIGS. 2A and 2B. The flange 70 is, in one embodiment, a unitary, homogenous extension of the wall 30, although in alternative embodiments can be separately formed and assembled to the tubular body 12. Regardless, the flange 70 extends radially from the proximal end 18, and defines a maximum outer diameter of the cover 10. Extension of the flange 70 forms a contact surface 72. The contact surface 72 is preferably planar, and establishes a known spacing of the cover 10, and in particular the cover tip 14, relative to the contact tonometer probe tip (not shown) upon final assembly. As described below, the cover 10 is preferably configured to correlate a longitudinal spacing between the cover tip 14 and the contact surface 72 of the flange 70 with a corresponding dimension of the tonometer probe tip. In one embodiment, the flange 70 has a substantially uniform thickness and does not include a longitudinal component. Further, for reasons described in greater detail below, to enhance rigidity of the flange 70, a thickness of the flange 70 is, in one embodiment, greater than a thickness of the wall 30 of the tubular body 12, for example at least 50% thicker.

Returning to FIGS. 1 and 2A, the intermediate section 44 generates a conical shape for the tubular body 12. For example, the outer surface 32 along the intermediate section 44 defines a reducing diameter in longitudinal extension from the proximal section 42 to the distal section 40. In one embodiment whereby the wall 30 has a substantially uniform thickness, the inner surface 34 defines an identical shape. The distal section 40 continues this conical shape, whereby the outer surface 32 decreases in diameter in longitudinal extension to the distal end 16. Thus, in one embodiment, the outer surface 32 at the distal section 40 and the intermediate section 44 combine to define a continuously tapering diameter from the proximal section 42 to the distal end 16, forming the tubular body 12 to a bullet-like shape. In one embodiment, the outer surface 32 along the intermediate section 44 and the distal section 40 defines a radius of curvature in the range of 1.0-1.5 inches, although other dimensions are equally acceptable. Further, the proximal section 42, in one embodiment, continues this conical shape, whereby the transition region 54 (FIG. 2B) defines a decreasing diameter in longitudinal extension from the second point 58 to the intermediate section 44. Thus, the outer surface 32 of the distal section 40, the intermediate section 44, and the proximal section 42 combine to define a continuously tapering diameter from the radial channel 52 to the distal end 16. In one embodiment, a thickness of the wall 30 and the selected material and method of forming is such that the tubular body 12 is configured to independently maintain the above-described bullet-like shape. Where the wall 30 is of a substantially uniform thickness, then, a maximum inner diameter of the tubular body 12 (i.e., a diameter defined by the inner surface 34) is defined at the radial channel 52.

As described above, in one embodiment, the cover tip 14 is preferably integrally, homogenously formed with the tubular body 12, although can be separately formed and assembled. Regardless, and as best shown in FIG. 2C, the cover tip 14 has a reduced thickness as compared to the wall 30. For example, a thickness of the cover tip 14 is an order of magnitude less than a thickness of the wall 30, and is in the range of 0.0005-0.0015 inch. With this construction, then, the tubular body 12 rigidly maintains an overall shape of the cover 10, whereas the cover tip 14 is highly thin so as to not interfere with operation of the tonometer probe tip (not shown).

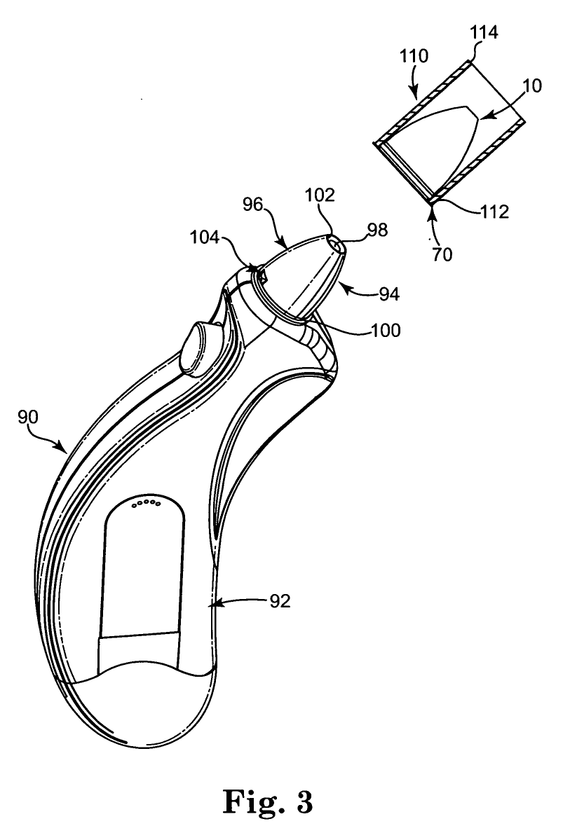

Assembly of the disposable cover 10 to a contact tonometer 90 is described with reference to FIG. 3. The contact tonometer 90 can assume a wide variety of forms, and generally includes a housing 92 maintaining a probe tip 94 as well as a number of other components not otherwise shown, such as electronic circuits, power supply, external lights, controller(s)/actuator(s), etc. The probe tip 94 includes a frame 96 maintaining a sensor 98 (referenced generally) and an annular rim 100. The frame 96 can be adapted for assembly to the housing 92 in a variety of fashions whereby the frame 96 extends from the housing 92, and terminates at a distal end 102. The rim 100 extends radially outwardly from a proximal end of the frame 96 and provides a stop surface for facilitating proper insertion of the cover 10, and can alternatively be provided as part of the housing 92. In another alternative embodiment, the rim 100 can be eliminated. Regardless, the frame 96 forms a radially-extending engagement feature 104 adjacent a proximal end thereof. The engagement feature 104 can assume a variety of forms, and in one embodiment includes two or more circumferentially-spaced radial protrusions (one of which is shown in FIG. 3). Alternatively, the engagement feature 104 can extend about an entire circumference of the frame 96, forming a circumferential rib. As described in greater detail below, the engagement feature 104 is captured within the radial channel 52 (FIG. 2A) of the cover 10 upon final assembly.

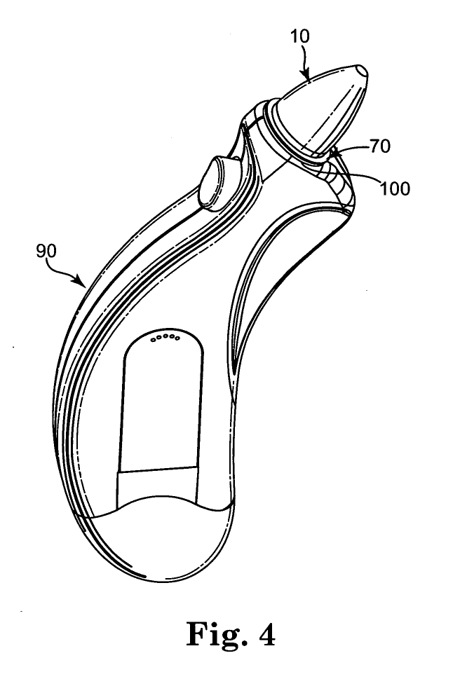

With the above in mind, assembly of the cover 10 over the probe tip 94 can be facilitated with an insertion tool 110. The insertion tool 110 can assume a variety of forms, but in one embodiment generally comprises a cylindrical tube having first and second ends 112, 114, and an inner diameter commensurate with an outer diameter of the tubular body 12 at the radial channel 52 (FIG. 2A). Assembly of the cover 10 includes placing the cover 10 within the insertion tool 110 as shown, with the first end 112 of the insertion tool 110 abutting the flange 70. The operator (not shown) then manipulates the insertion tool 110 to position the cover 10 over the probe tip 94. A force is applied to the insertion tool 110, forcing the flange 70, and thus the cover 10, over the probe tip 94, and in particular the engagement feature 104. Thus, the insertion tool 110 assists in focusing an insertion force onto the flange 70. Alternatively, the cover 10 can be assembled over the probe tip 94 without the use of the insertion tool 110. Upon final assembly, the contact surface 72 (FIG. 2B) of the flange 70 abuts the rim 100 of the tonometer 90 as shown in FIG. 4.

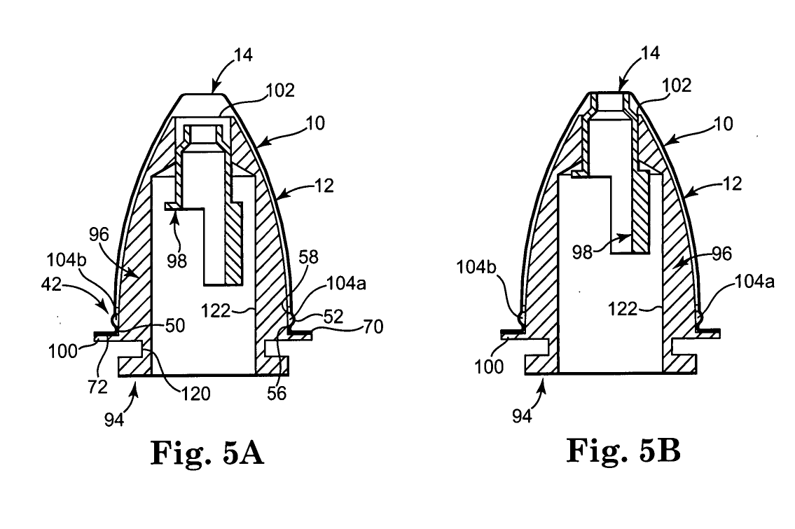

The assembled position of the cover 10 relative to the probe tip 94 is better illustrated in FIGS. 5A and 5B. As a point of reference, the housing 92 (FIG. 3) is omitted from the view of FIGS. SA and 5B for ease of illustration, and the rim 100 is shown generally. It will be understood, however, that in one embodiment, the frame 96 of the probe tip 94 forms a groove 120 that otherwise facilitates attachment to the housing 92. In addition to the frame 96, FIGS. 5A and 5B schematically illustrate the sensor 98 of the probe tip 94. The sensor 98 can assume a form appropriate for performing a contact tonometer operation and is connected to other components (e.g., circuit board) of the tonometer 90 (FIG. 3) via wiring (not shown). Regardless, the sensor 98 is received and maintained within a longitudinal aperture 122 defined by the frame 96 such that the sensor 98 can move (e.g., slide) relative to the frame 96. In particular, the aperture 122 extends from the distal end 102 of the frame 96, and guides the sensor 98 between a deactivated position (FIG. 5A) and an activated position (FIG. 5B). In the deactivated position of FIG. 5A, the sensor 98 is entirely recessed within the aperture 122 of the frame 96. In the activated state of FIG. 5B (i.e., when measuring intraocular pressure), a portion of the sensor 98 extends distally beyond the distal end 102 of the frame 96.

With the above in mind, and with specific reference to FIG. 5A, upon final assembly, the engagement feature 104 (illustrated as spaced radial projections 104a, 104b in FIG. 5A), is captured within the radial channel 52 of the cover 10. More particularly, an inner diameter of the tubular body 12 at the first and second points 56, 58 of the proximal section 42 is less than a diameter defined by the engagement feature 104a, 104b, such that the tubular body 12 physically engages the engagement feature 104a, 104b at the first and second points 56, 58. In addition, and in one embodiment, the reduced inner diameter associated with the base region 50 of the tubular body 12 ensures that the cover 10 will not unexpectedly dislodge distally from the probe tip 94. Conversely, interface between the contact surface 72 of the flange 70 and the rim 100 ensures that the engagement feature 104a, 104b is fully captured within the radial channel 52. That is to say, a longitudinal spacing between the contact surface 72 of the flange 70 and the radial channel 52 corresponds with a longitudinal spacing between the rim 100 and the engagement feature 104a, 104b. Regardless, the tubular body 12 is sufficiently rigid to maintain the overall conical shape of the cover 10 in the deactivated state of the probe tip 94. The tubular body 12 does not inherently sag or buckle against the distal end 102 of the frame 96 such that the spacing between the cover tip 14 and the distal end 102 of the frame 96 is consistently maintained.

By independently maintaining a known spacing between the cover tip 14 and the distal end 102 of the frame 96, the disposable cover 10 virtually eliminates possible interference with operation of the probe tip 94. In particular, and in one embodiment, the cover 10 is configured such that a longitudinal spacing between the contact surface 72 of the flange 70 and the cover tip 14 corresponds with the longitudinal spacing between the rim 100 and the sensor 98 in the activated state. This relationship is shown in FIG. 5B whereby when the probe tip 94 performs a contact tonometer operation, the sensor 98 presses against the cover tip 14. Due to the inherent characteristic of the cover 10 whereby the tubular body 12 independently maintains the conical shape shown (that otherwise corresponds with the conical shape of an outer surface of the frame 96), the cover 10 does not overtly resist distal movement of the sensor 98 in a manner that might otherwise produce erroneous force readings by the sensor 98. As the sensor 98 transitions from the deactivated state of FIG. 5A to the activated state of FIG. 5B, the sensor 98 is not required to force the cover 10 away from the distal end 102 of the frame 96, as might otherwise occur if the tubular body 12 naturally collapsed toward the distal end 102.

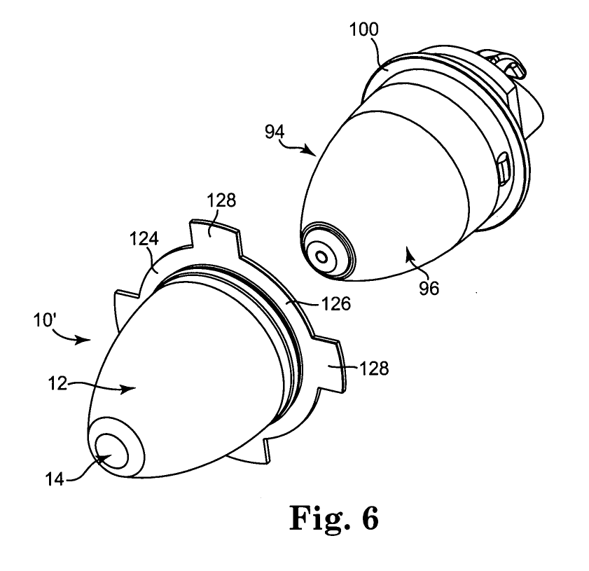

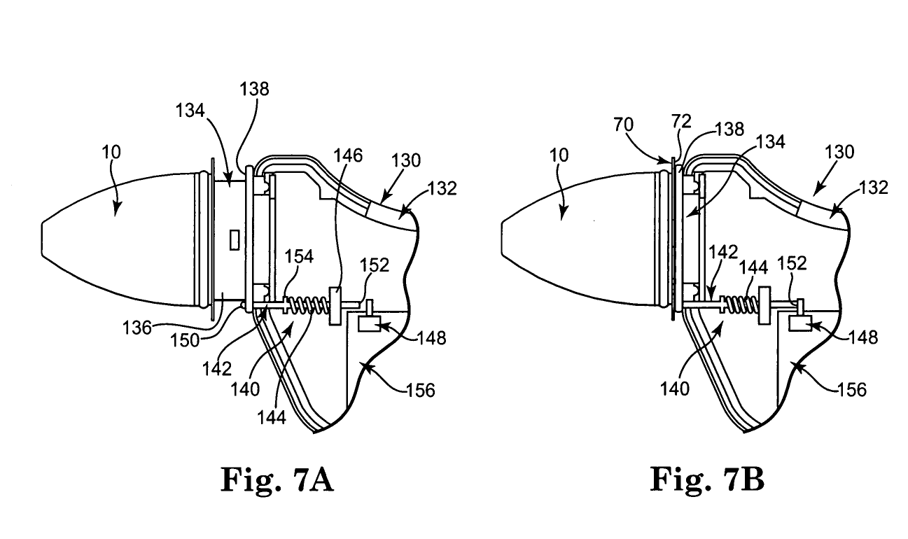

The cover 10 can be removed from the probe tip 94 by grasping and manipulating the flange 70 and/or the tubular body 12 away from the frame 96. To facilitate easier removal, and with reference to FIG. 6, an alternative embodiment cover 10′ includes a flange 124 adapted to provide a grasping surface. The cover 10′ is highly similar to cover 10 (FIG. 1) previously described, and includes the tubular body 12, the cover tip 14, and the flange 124. The flange 124 radially extends from the tubular body 12 opposite the cover tip 14, and includes, in one embodiment, a base 126 and one or more tabs 128 (four of which are shown in FIG. 6). The base 126 extends to a first diameter from the tubular body 12. The tab(s) 128 radially extend from the base 126 to define a second diameter that is greater than the first diameter of the base 126. In particular, extension of the tab(s) 128 defines a diameter greater than that of the rim 100 associated with the probe tip 94, such that when the cover 10′ is disposed over the probe tip frame 96, the tab(s) 128 extend radially beyond the rim 100. With this configuration, then, the tab(s) 128 provide a readily available grasping surface for a user to assist in removing the cover 10′ from the probe tip 94. While four, equidistantly-spaced tabs 128 are illustrated in FIG. 6, any other number (lesser or greater) or arrangement is also acceptable.

Embodiments of the disposable cover of the present invention provide a marked improvement over previous designs. In combination with a design of the probe tip, the cover facilitates simply yet consistently stable fixation of the cover over the probe tip. Further, the cover minimizes the interference, if any, with operation of the probe tip.

In one alternative embodiment, the cover 10 and the contact tonometer are further configured to provide a user with a confirmation that the cover 10 has been properly assembled to the probe tip. For example, FIG. 6A illustrates the cover 10 partially assembled to an alternative embodiment contact tonometer 130. Once again, the contact tonometer 130 can assume a variety of forms and generally includes a housing 132 maintaining a probe tip 134. The probe tip 134 includes frame 136 maintaining a sensor (not shown), and a rim 138. Alternatively, the rim 138 can be an integral part of the housing 132. Regardless, the contact tonometer 130 further includes a cover detection device 140. The detection device 140 can assume a variety of forms, and in one embodiment includes a plunger 142, a biasing device (e.g., spring) 144, a support 146, and a switch 148. The support 146 maintains the plunger 142 relative to the housing 132 such that a first end 150 of the plunger 142 extends distally beyond the rim 138, whereas a second end 152 of the plunger 142 is positioned adjacent the switch 148. The biasing device 144 is positioned between the support 146 and a flange 154 provided by the plunger 142, biasing the plunger 142 to the open position of FIG. 6A. In the open position, the second end 152 is spaced from the switch 148 that is otherwise electronically connected to primary circuitry 156 (shown generally) of the tonometer 130. Thus, in the open position, the switch 148 is not acted upon by the plunger 142, thus providing a signal (or an absence of a signal) indicative of the cover 10 not being fully assembled to the probe tip 134.

When the cover 10 is properly inserted over the probe tip 134, the detection device 140 operates to signal (actively or passively) the main circuitry 156 with information indicative of proper assembly. For example, as shown in FIG. 6B, when the cover 10 is fully assembled, the contact surface 72 of the flange 70 presses against the first end 150 (hidden in FIG. 6B; shown in FIG. 6A) of the plunger 142. When sufficient force is placed upon the flange 70 to overcome the bias of the biasing device 144, the plunger 142 is forced from the open state of FIG. 6A to the closed state of FIG. 6B in which the second end 152 of the plunger 142 contacts or engages the switch 148. In response to this interface, the switch 148 provides a signal (or lack of a signal) to the main circuitry 156 indicative of the cover 10 being properly assembled over the probe tip 134. More particularly, in the open state (FIG. 6A), a spacing between the second end 152 of the plunger 142 and the switch 148 corresponds with a spacing between the first end 150 of the plunger 142 and the rim 138. Thus, when the contact surface 72 of the flange 70 contacts the rim 138, the second end 152 of the plunger 142 contacts the switch 148. Alternatively, the detection device 140 can assume a wide variety of other forms.

Although the present invention has been described with reference to preferred embodiments, workers skilled in the art will recognize that changes can be made in form and detail without departing from the spirit and scope of the claims.

What is claimed is:

1. A disposable cover for a probe tip of a contact tonometer, the cover comprising:
a tubular body defined by a wall having an outer surface and an inner surface, the tubular body defining:
a distal section terminating at a distal end,
a proximal section opposite the distal end and terminating in an open, proximal end, wherein the inner surface along the proximal section forms a radial channel longitudinally spaced from the proximal end, the radial channel being configured to mate with a corresponding feature of a probe tip and characterized by an increased diameter as compared to an inner diameter of the tubular body immediately adjacent to the radial channel at either side thereof,
an intermediate section extending between the distal and proximal sections, wherein the outer surface along the intermediate section defines a conical shape, decreasing in diameter from the proximal section to the distal section; and
a cover tip formed at the distal end such that the cover is closed at the distal end.

2. The disposable cover of claim 1, further comprising a flange radially extending from the proximal end.

3. The disposable cover of claim 2, wherein the flange and the tubular body are homogenously formed.

4. The disposable cover of claim 2, wherein the flange has a substantially uniform thickness.

5. The disposable cover of claim 2, wherein the flange defines a maximum outer diameter of the cover.

6. The disposable cover of claim 2, wherein the flange includes:
a base radially extending from the proximal end of the tubular body to a first diameter; and
at least one tab radially extending from the base to define a second diameter greater than the first diameter.

7. The disposable cover of claim 1, wherein the wall has a substantially uniform thickness.

8. The disposable cover of claim 1, wherein the cover tip and the tubular body are homogenously formed.

9. The disposable cover of claim 1, wherein the radial channel defines a maximum inner diameter of the tubular body.

10. The disposable cover of claim 1, wherein the inner surface of the wall along the radial channel defines a longitudinal curve extending from a first point adjacent the proximal end to a second point opposite the proximal end, the curve increasing in diameter from the first point to a middle region, and decreasing in diameter from the middle region to the second point, and further wherein an inner diameter of the tubular body at the first point is less than an inner diameter of the tubular body at the second point.

11. The disposable cover of claim 10, wherein the proximal section further defines a base region longitudinally extending from the first point to the proximal end and a transition region longitudinally extending from the second point to the intermediate section, and further wherein an inner diameter of the base region is less than an inner diameter of the transition region.

12. The disposable cover of claim 10, wherein the proximal section further defines a base region longitudinally extending from the first point to the proximal end, and further wherein the inner surface defines a uniform diameter in longitudinal extension along at least a majority of the base region.

13. The disposable cover of claim 1, wherein the outer surface at the distal section and the intermediate section combine to define a continuously tapering diameter from the proximal section to the distal end.

14. The disposable cover of claim 13, wherein the outer surface at the distal section, intermediate section, and proximal section combine to define a continuously tapering diameter from the radial channel to the distal end.

15. The disposable cover of claim 1, wherein the cover is configured to independently maintain the conical shape of the intermediate section.

16. A combination contact tonometer and disposable cover, the combination comprising:
a contact tonometer including a probe tip; and
a disposable cover comprising:
a tubular body defined by a wall having an outer surface and an inner surface, the tubular body defining:
a distal section terminating at a distal end,
a proximal section opposite the distal end and terminating in an open, proximal end, wherein the inner surface along the proximal section forms a radial channel longitudinally spaced from the proximal end, the radial channel being characterized by an increased inner diameter as compared to an inner diameter of the tubular body immediately adjacent to the radial channel at either side thereof,
an intermediate section extending between the distal and proximal sections, wherein the outer surface along the intermediate section defines a conical shape, decreasing in diameter from the proximal section to the distal section;
a cover tip formed at the distal end such that the cover is closed at the distal end;
wherein the combination tonometer and cover are configured such that the cover is removable secured over the probe tip by capturing a feature of the probe tip within the radial channel.

17. The combination contact tonometer and disposable cover of claim 16, wherein the probe tip includes a frame maintaining a sensor, the housing including a radially extending engagement feature adjacent a proximal end thereof, and further wherein the combination tonometer and cover are configured such that in an assembled state, the engagement feature nests within the radial channel.

18. The combination contact tonometer and disposable cover of claim 17, wherein in the assembled state, the radial channel engages opposing upper and lower surfaces of the engagement feature.

19. The combination contact tonometer and disposable cover of claim 16, wherein the frame defines a proximal end and a distal end, and further wherein the sensor is slidably maintained within the housing to provide an activated position in which a portion of the sensor projects distally from the distal end of the frame and a deactivated position in which the sensor is recessed within the frame, and further wherein the cover is configured to maintain a spacing between the cover tip and the distal end of the frame upon assembly of the cover over the probe tip with the sensor in the deactivated position.

20. The combination contact tonometer and disposable cover of claim 19, wherein the tonometer further includes a detection device for detecting a position of the cover relative to the probe tip.
